(19) United States
(12) Patent Application Publication
(10) Pub. No.: US 2018/0296093 A1
(43) Pub. Date: Oct. 18, 2018

(54) Apparatus and Method for Diagnosing Vessel Occlusion

(71) Applicant: Yince Loh, Seattle, WA (US)

(72) Inventor: Yince Loh, Seattle, WA (US)

(21) Appl. No.: 15/828,840

(22) Filed: Dec. 1, 2017

Related U.S. Application Data
(60) Provisional application No. 62/486,177, filed on Apr. 17, 2017.
(60) Provisional application No. 62/517,459, filed on Jun. 9, 2017.

(51) Int. Cl. A61B 5/00; A61B 5/026; A61B 8/06; A61B 8/00; A61B 8/08

(52) U.S. Cl. CPC A61B 5/0042; A61B 5/0261; A61B 5/6803; A61B 5/684; A61B 8/4254; A61B 8/4209; A61B 8/488; A61B 5/7282; A61B 8/5223; A61B 8/06

(57) ABSTRACT

Apparatus and methods for diagnosing conditions consistent with the presence of cranial blood vessel blockage and large vessel occlusions (LVO's) using the framework of a small head-harness that is transportable, field-expedient, and durable. A single pulse set using ultrasonic or near-infrared energy is broadcast into a patient's brain allowing the apparatus to perform an area scan of the brain and detect and decipher cranial blood vessel blockage and LVO signal patterns. The interpretation of the pattern lies within the internal programming which produces a binary signal as to whether an LVO is suspected or not.

### CROSS-REFERENCES TO RELATED APPLICATIONS

This application claims priority to U.S. Provisional Application No. 62/486,177, filed Apr. 17, 2017, and U.S. Provisional Application No. 62/517,459, dated Jun. 9, 2017, the disclosures of which are incorporated by reference herein in their entirety.

### STATEMENT REGARDING FEDERALLY SPONSORED RESEARCH OR DEVELOPMENT

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND

Nearly 800,000 strokes occur in the US annually, and almost 3 million Americans are currently disabled from them. Stroke is the third leading cause of death in the US and is the leading cause of disability costing over $73 billion; year in the US alone.

The most disabling and deadly ischemic strokes (i.e. lack of blood flow to the brain) result from large vessel occlusions (LVO's). Patients with LVO's have extremely poor outcomes without treatment and until recently, respond poorly to standard of care (tissue plasminogen activator, or tPA). In the 1990s, the MERCI retrieval system marked the advent of an endovascular method and system that could be used to remove clot from the brain vessels. Several decades later, the second and third generation devices furthered the concept that clot the brain vessels could be extracted using devices and catheters inserted through the groin artery. Within one year, five randomized controlled trials all showed a positive benefit of endovascular therapy (EVT) over optimal medical management of LVO's. All had a time limit for inclusion in the study and several showed that earlier intervention produced better clinical outcomes.

In one of these studies, patients transferred to a hospital without ENT capability had an average delay of two hours before arriving to the final EVT-capable facility. This is an unacceptable delay when time is critical to preserving brain function. State health departments, National Accreditation Organizations, and systems of care designers have implemented designations for stroke capabilities to distinguish those capable of providing standard of non-EVT stroke care and those with 24/7 EVT capability. Emergency medical systems (EMS) will be integral in appropriate patient triage and delivery to stroke centers, much like trauma triage. The emerging dilemma now lies in accuratefield stroke triage. Only a portion of ischemic strokes result from LVO's, and EVT does not benefit the rest. Movement of both LVO and non-LVO stroke patients to a single EVT-capable center would potentially delay or deprive a patient of standard of care treatment for non-LVO strokes. It would potentially also overwhelm the EVT-capable hospital.

Imaging identification of LVO's already exists with MRI and CT. The former is not feasible for field deployment, while the field-deployable versions of the latter are extremely expensive and likely to be a limited yet paradoxically under-utilized resource. Transcranial Doppler (TCD) ultrasonography and near infrared scanners are portable tools that can identify LVO's, but are operator-dependent.

There is a need to diagnose LVOs quickly and provide appropriate medical intervention. The ideal adjunct to the EMT or paramedic assessing a possible stroke patient is a field-expedient, operator-independent device to help determine whether a patient, potentially needs EVT. Such a device could effectively diagnose while minimizing diagnostic error and operator training. Such a device could also help emergency physicians at non-EVT hospitals identify EVT-eligible patients earlier and expedite transfer to EVT-capable hospitals without doing additional time-consuming imaging.

### SUMMARY

An objective of this invention is to provide apparatus and methods for diagnosing conditions consistent with the presence of blockage patient's cranial blood vessels, including the presence of LVO's, using the framework of a small head-harness that is transportable, field-expedient, and durable. A single pulse set using ultrasonic or near-infrared energy is broadcast into the patient's brain allowing the apparatus to perform an area scan of the brain and detect and decipher cranial blood vessel blockage and LVO signal patterns. The interpretation of the pattern lies within the internal programming which produces a binary signal as to whether an LVO is suspected.

### DRAWINGS DESCRIPTION

Other features and advantages of the present invention will become apparent in the following detailed descriptions of certain preferred embodiments with reference to the accompanying drawings, of which:

FIG. 1 is an elevation view of a small, lightweight, self-contained, portable, ruggedized, head-mounted diagnostic tool with a transducer array and other system components useful for diagnosing conditions consistent with the existence of large vessel occlusion according to illustrative embodiments of the present invention;

FIG. 2A is an elevation view of a small, lightweight, self-contained, portable, ruggedized, head-mounted diagnostic tool with a transducer array and other system components useful for diagnosing conditions consistent with the existence of large vessel occlusion according to illustrative embodiments of the present invention;

FIG. 2B is a plan view of a transducer array useful for diagnosing conditions consistent with the existence of large vessel occlusion according to illustrative embodiments of the present invention;

FIG. 2C is a side view of a transducer array illustrating one example of how energy from the transducer array is transmitted into a patient's brain using a single pulse set according to illustrative embodiments of the present invention;

FIG. 2D is a side view of a transducer array illustrating one example of how energy from the transducer array is transmitted into a patient's brain using a single pulse set according to illustrative embodiments of the present invention;

FIG. 2E is a side view of a transducer array illustrating one example of how energy from the transducer array is transmitted into a patient's brain using a single pulse set according to illustrative embodiments of the present invention;

FIG. 3 is an illustration of one example of how data collected from a first portion of the brain is compared to that collected from a second portion of the brain wherein sufficient differences are identified to suggest the presence of LVO's in the patient's brain according to illustrative embodiments of the present invention;

FIG. 4 shows a method of diagnosing conditions consistent with the existence of large vessel occlusion by performing an area scan of a patient's brain and comparing data collected from a first portion of a patient's brain with that collected from a second portion of a patient's brain according to illustrative embodiments of the present invention;

FIG. 5 shows a method of diagnosing conditions consistent with the existence of large vessel occlusion by performing an area scan of a patient's brain using transcranial ultrasound and comparing data collected from a first portion of a patient's brain with that collected from a second portion of a patient's brain according to illustrative embodiments of the present invention;

FIG. 6 shows a method of diagnosing conditions consistent with the existence of large vessel occlusion by performing an area scan of a patient's brain using near infrared imaging and comparing data collected from a first portion of a patient's brain with that collected from a second portion of a patient's brain according to illustrative embodiments of the present invention; and

FIG. 7 shows a method of diagnosing conditions consistent with the existence of large vessel occlusion by performing an area scan of a patient's brain using transcranial ultrasound, comparing data collected from a first portion of a patient's brain with that collected from a second portion of a patient's brain, and treating suspected LVO using transcranial doppler energy according to illustrative embodiments of the present invention.

### DESCRIPTION

In the Background, Summary, and Drawings Description above, in the Description and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e. contain only) components A, B, and C, or can contain not only components A, B, and C but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)—(a second number),” this means a range whose lower limit is the first number and whose upper limit is the second number. For example, 25 to 100 mm means a range whose lower limit is 25 mm, and whose upper limit is 100 mm.

The term “area scan” is used herein to reference the act of looking at all parts of something in order to detect a feature by means of causing a part of the body to be traversed by a detector beam.

The present invention is related to a small, lightweight, self-contained, portable, ruggedized, head-mounted diagnostic tool for diagnosing conditions consistent with the existence of blockage in a patient's cranial blood vessels, including large vessel occlusions, and methods of diagnosing conditions consistent with the existence of blockage in a patient's cranial blood vessels, including large vessel occlusions. Multiple embodiments of the invention are described hereinafter with reference to the accompanying drawings. This invention may, however, be embodied in many different forms and should not be construed as limited to the embodiments set forth herein; rather, these embodiments are provided so that this disclosure will be thorough and complete and will fully convey the scope of the invention to those skilled in the art.

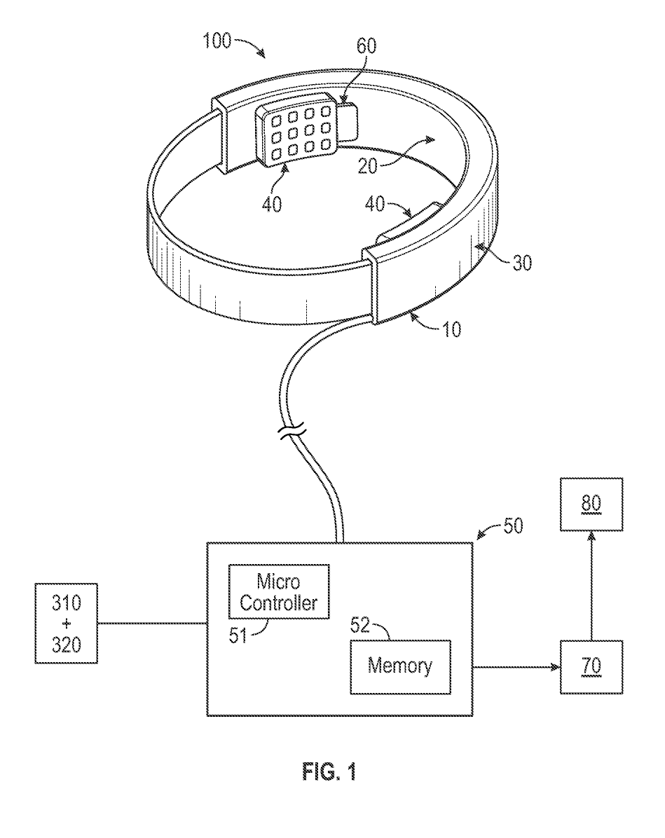

Described herein is a diagnostic apparatus 100 which utilizes scanning technologies like transcranial Doppler ultrasound and near infrared imaging, and methods for their use to diagnose blockage in a patient's cranial blood vessels, including LVOs. According to an embodiment, referring to FIG. 1, the apparatus 100 is comprised of a headset 10. Preferably, the headset 10 is adjustable so as fit the cranium of more than one patient.

Using the framework of a small head-harness that is transportable, field-expedient, and durable, a single pulse set using ultrasonic or near-infrared energy is broadcast into the patient's brain allowing the apparatus to perform an area scan of the brain and detect and decipher cranial blood vessel blockage and LVO signal patterns. The interpretation of the pattern lies within the internal programming which produces a binary signal as to whether an LVO is suspected or not.

According to an embodiment,e headset 100 is comprised of an interior side 20 and an exterior side 30. According an embodiment, a scanning device is mounted on the interior side 20 of the headset 10. According to an embodiment, the scanning device is comprised of at least one transducer 40 which is mounted on the interior side 20 of the headset 10. According to an embodiment, at least one array of transducers 40 is mounted to the interior side 20 of the headset 10. According to an embodiment, the interior side 20 is comprised of a plurality of transducers 40. According to an embodiment, the plurality of transducers 40 are arrayed. According to an embodiment, each transducer 40 is a non-focused ultrasound transducer. According to an embodiment, each transducer 40 is a non-focused near infrared transducer. According to an embodiment, each transducer 40 or transducer array 40 is mounted to the interior side 20 of the headset 10. According to an embodiment, at least one mounted transducer 40 may be adjustably on the interior side 20 of the headset 10.

According to an embodiment, the scanning device is comprised of at least two transducer arrays 40 where at least one transducer array 40 is configured to align with the left temple of a human patient; and at least o transducer array is configured to align with the right temple of a human patient. According to an embodiment,the apparatus 100 is further comprised of an electronic circuit 50. According to an embodiment, the electronic circuit 50 is operably connected to the headset 10. According to an embodiment, the electronic circuit controls each transducer 10. According to an embodiment, the electronic circuit 50 is comprised of a microcontroller 51 and memory 52 which comprise digitally encoded instructions in non-volatile memory for autonomously driving at least one diagnostic operation.

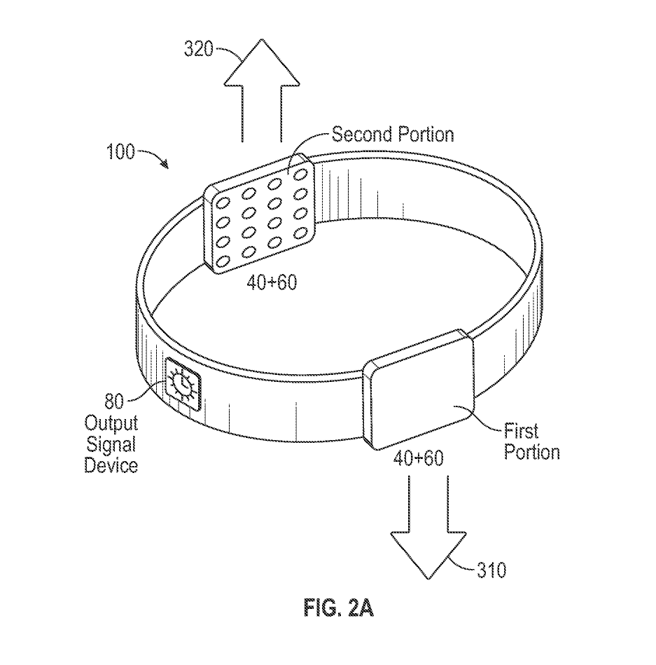

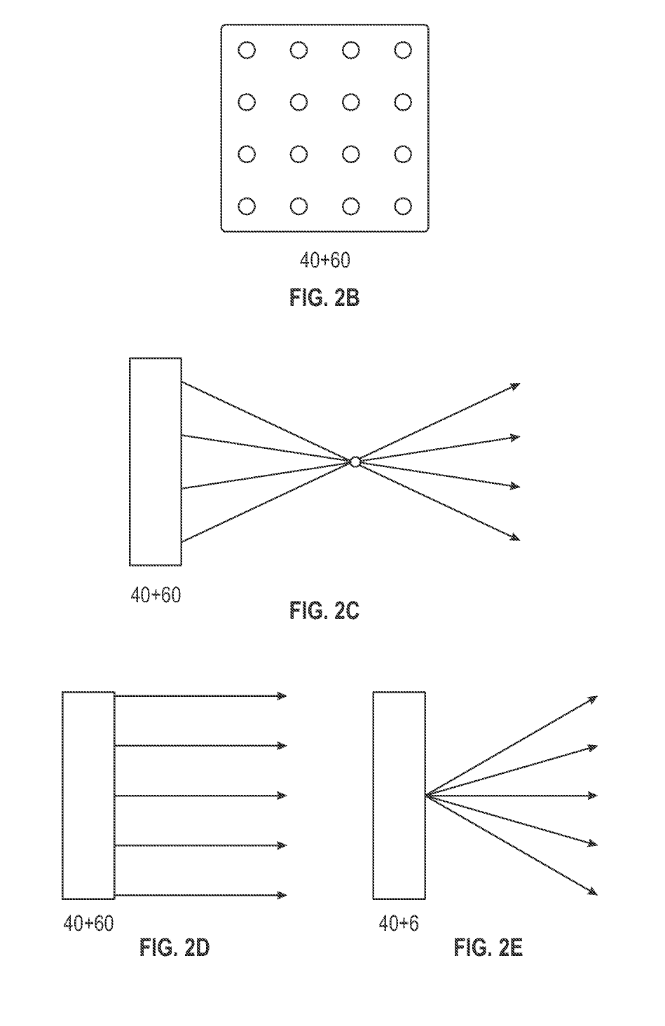

According to embodiments of the invention, referring to FIGS. 2B, 2C, 2D and 2E, the transducer array is configured to transmit energy into a patient's brain at varying angles of transmission 40. Reflected energy is detected by a sensor 60 which is proximately located or adjacent to at least one transducer 40.

Referred or reflected energy is detected by a sensor 60. According to an embodiment, the sensor 60 is proximately located or adjacent to at least one transducer 40. According to an embodiment the electronic circuit 50 is operably connected to the signal sensor 70. In an embodiment, the sensor is also operably connected to a signal interpreter 80. According to an embodiment, the signal sensor 70 and signal interpreter 80 are operably connected to the headset 10.

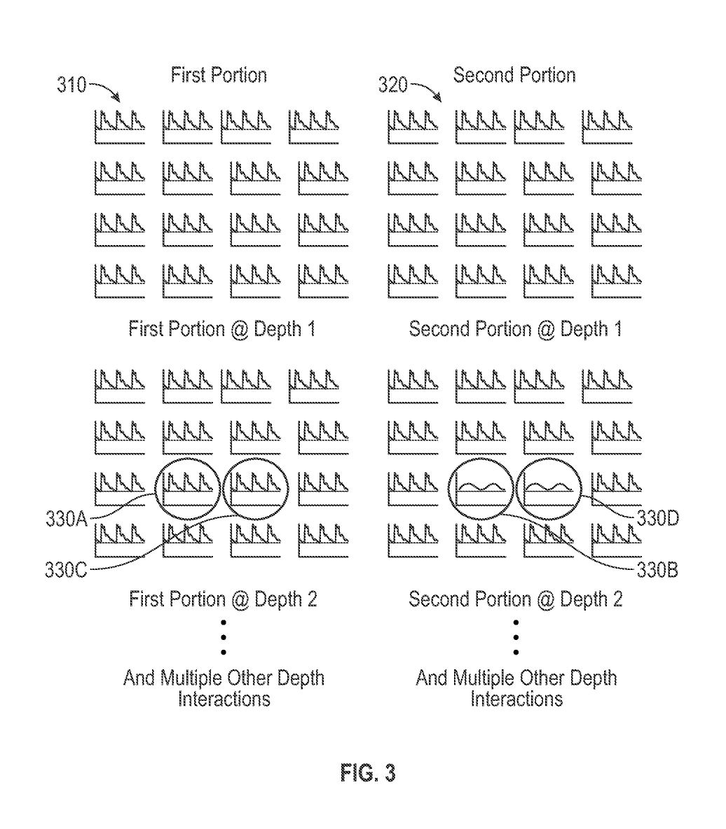

According to an embodiment of the invention, referring to FIG. 2A and FIG. 3, the digitally encoded instructions of electronic circuit 50 utilize the data collected by sensor 60 from a first side of the apparatus 100 (310) and data collected by sensor 60 from a second side of the apparatus 100 (320). Data s collected by the sensor 60 from a wide area of a patient's brain (FIGS. 2C, 2D, and 2E), with the data set collected from a first portion of the patient's brain compared to the data set collected from a second portion of that patient's brain (310, 320) within the electronic circuit 50. As a means of illustration, one possible example of a comparison is depicted in FIG. 3, comparing 330A to 330B, and 330C to 330D, wherein data collected is represented by nonspecific graphical waveforms. The programming of the digitally coded instructions its the electronic circuit 50 selects the most likely representative waveforms from the data collected (FIG. 3). If this process of comparing one data set to the corresponding data set identifies a deviation or variation in waveform, waveform set, or processed waveform set that exceeds a pre-determined threshold, a signal is transmitted to an output signal device 80.

According to an embodiment, a localizing device is mounted on the interior side 20 of the headset 10. An embodiment of the apparatus 100 utilizes the transducer or transducer array as the localizing device to identify and signal that a proper configuration of the harness has been achieved. In an embodiment, the proper configuration is signaled through a visual output display 80. An embodiment of the apparatus 100 operably combines the scanning device and the localizing device.

An embodiment of the apparatus 100 is configured to use a separate transducer or transducer array as the localizing device using near infrared spectrum energy and decipher referred or reflected energy to identify and signal that a proper configuration of the harness has been achieved. The sensor array of this embodiment of the invention is comprised of near infrared imaging transmitters and receivers. In an embodiment, the proper configuration is signaled through a visual output display 80. An embodiment of the apparatus 100 operably combines the scanning device and the localizing device.

An embodiment of the apparatus 100 is configured to use a separate sector imaging phased array and decipher referred or reflected sound to identify and signal that a proper configuration of the harness has been achieved. The sensor array of this embodiment of the invention is comprised of a sector imaging phased array.

An embodiment of the apparatus 100 is configured to provide an ultrasound frequency 510 to a human patient and decipher referred or reflected energy (520, 530) to diagnose LVO (540). In an embodiment, the apparatus 100 delivers ultrasound frequency 510) between 1 and 5 MHz. According to an embodiment, the apparatus 100 delivers ultrasound frequency (510) between 1.5 and 2.5 MHz. According to an embodiment, the ultrasound frequency may be delivered (510) as a constant wave. According to an embodiment, the ultrasound frequency may be delivered (510) as a pulse.

An embodiment of the apparatus 100 is configured to provide near infrared spectrum energy to a human patient and decipher referred or reflected energy to diagnose LVO (610, 620, 630, 640). The sensor array of this embodiment of the invention is comprised of near infrared imaging transmitters and receivers.

According to an embodiment, the signal interpreter 70 examines and processes the detected energy signal patterns through deconvolution calculations. According to an embodiment, these patterns are represented through a visual output display 80 to signal whether an LVO is detected (440, 540, 640, 740).

According to an embodiment, the interior side 20 of the headset a attach to individually packaged, individual use, disposable pads that improve the transduction and sensing of signals.

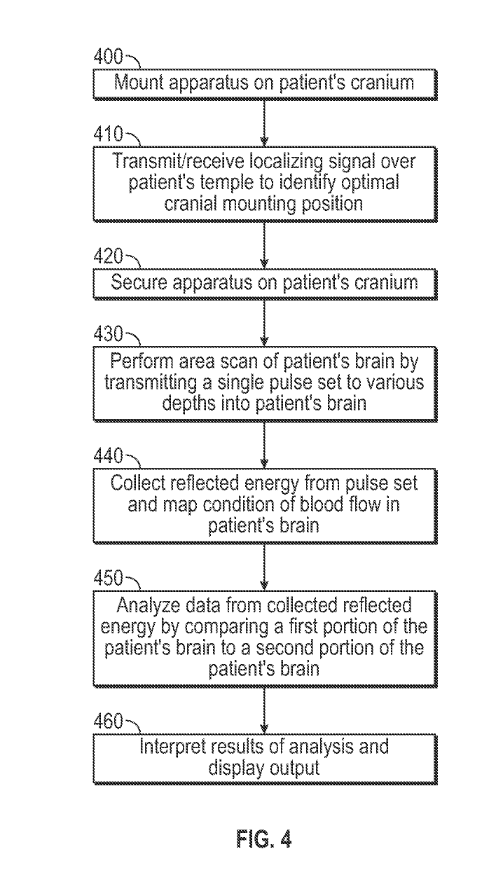

Referring to the method described in FIG. 4, an apparatus employing scanning technology is mounted to the head of a patient (400, 410, 420) so that the scanner's transmitters and receivers (40 and 60) are situated adjacent to the temples of a patient's head. According to an embodiment, proper contact between the scanning device and the patient's cranium is ensured using an appropriate insertional pad. A single pulse set is broadcast into the patient's brain allowing the apparatus to perform an area scan of the brain 430. Data is generated using reflected waves produced by each single pulse within a set which are collected by the receivers of the scanning apparatus 440. The status of blood flow in the patient's brain is analyzed by comparing the data collected from a first portion of the patient's brain to that collected from a second portion of the patient's brain 450. A diagnosis is developed based upon the analysis described (FIG. 3) with feedback provided to users of the head-mounted scanning apparatus 460 by way of the signal output device 80.

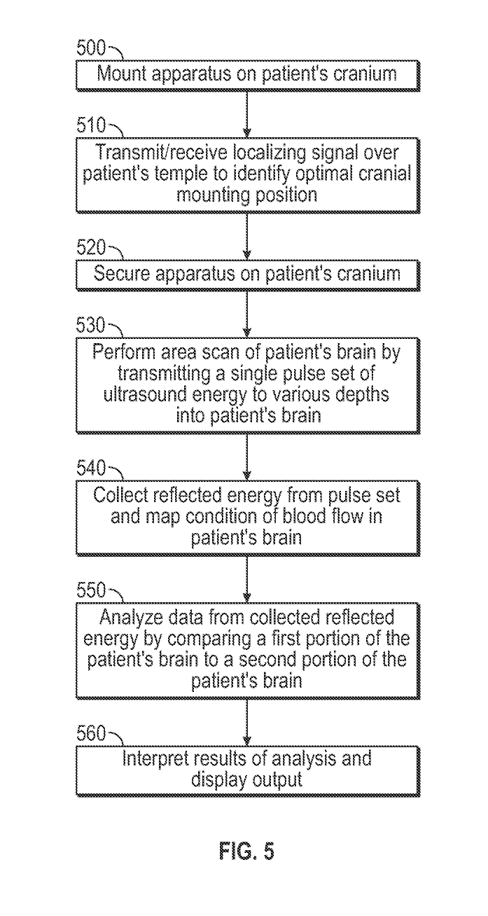

Referring to the method described in FIG. 5, an apparatus employing transcranial ultrasound scanning technology is mounted to the head of a patient (500, 510, 520) so that the scanner's transmitters and receivers (40 and 60) are situated adjacent to the temples of a patient's head. According to an embodiment, proper contact between the scanning device and the patient's cranium is ensured using an appropriate insertional pad. A single pulse set is broadcast into the patient's brain allowing the apparatus to perform an area scan of the brain 530. Data is generated using reflected waves produced by each single pulse within a set which are collected by the receivers of the scanning apparatus 540. The status of blood flow in the patient's brain is analyzed by comparing the data collected from a first portion of the patient's brain to that collected from a second portion of the patient's brain 550. A diagnosis is developed based upon the analysis described (FIG. 3) with feedback provided to users of the head-mounted scanning apparatus 560 by way of the signal output device 80.

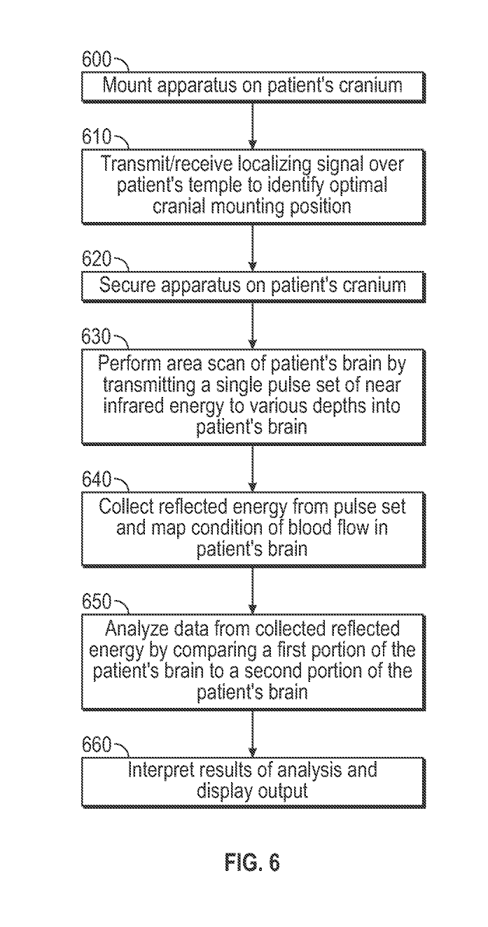

Referring to the method described in FIG. 6, an apparatus employing near infrared scanning technology is mounted to the head of a patient (600, 610, 620) so that the scanner's transmitters and receivers (40 and 60) are situated adjacent to the temples of a patient's head. According to an embodiment, proper contact between the scanning device and the patient's cranium is ensured using an appropriate insertional pad. A single pulse set is broadcast into the patient's brain allowing the apparatus to perform an area scan of the brain 630, and generating a data set using reflected waves collected by the receivers of the scanning apparatus 640. The status of blood flow in the patient's brain is analyzed by comparing the data set collected from a first portion of the patient's brain to a data set collected from a second portion of the patient's brain 650. A diagnosis is developed based upon the analysis described (FIG. 3) with feedback provided to users of the head-mounted scanning apparatus 660 by way of the signal output device 80.

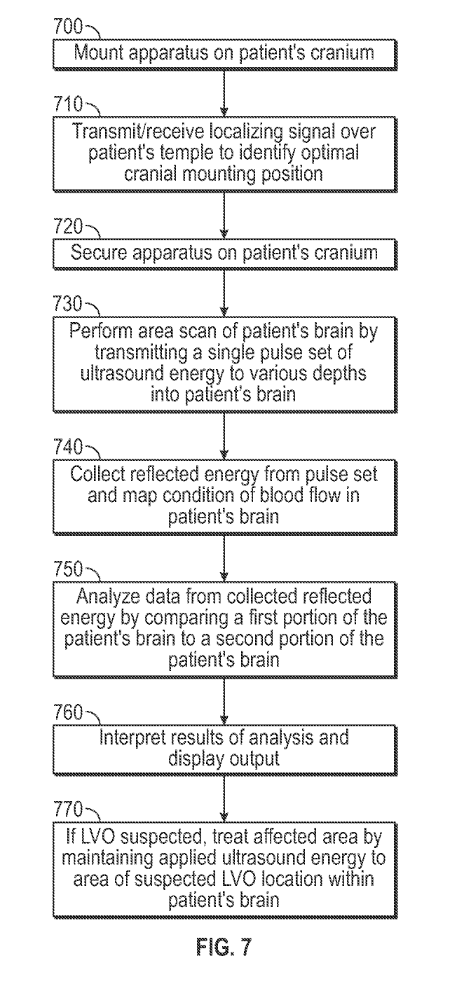

Referring to the method described in FIG. 7, an apparatus employing transcranial ultrasound scanning technology is mounted to the head of a patient (700, 710, 720) so that the scanner's transmitters and receivers (40 and 60) are situated adjacent to the temples of a patient's head. According to an embodiment, proper contact between the scanning device and the patient's cranium is ensured using an appropriate insertional pad. A single pulse set is broadcast into the patient's brain allowing the apparatus to perform an area scan of the brain 730 and generating a data set using reflected waves collected by the receivers of the scanning s 740. The status of blood flow in the patient's brain is analyzed by comparing the data set collected from a first portion of the patient's brain to a data set collected from a second portion of the patient's brain 750. A diagnosis is developed based upon the analysis described (FIG. 3) with feedback provided to users of the head-mounted scanning apparatus 760 by way of the signal output device 80. If conditions consistent with large vessel occlusion are found to exist, treatment is initiated by targeting the impacted area, applying and maintaining focused ultrasound energy on the suspected LVO within he patient's brain 770.

1. A head-mounted diagnostic tool for diagnosing conditions consistent with the existence of blockage of a patient's cranial blood vessels, such as large vessel occlusions, comprising:
a. a power source;
b. at least one scanning device for scanning a patient's brain, characterized in that the scanning device is stationary relative to the patient's cranium, and further comprising a transducer array incorporating a transmitter array and a receiver array;
c. at least one localizing device for indicating proper head placement;
d. a data processing device for analyzing and interpreting results generated by the scanning device;
e. an output device; and
f. an adjustable support configured to: mount the scanning device, the localizing device, the data processing device, and the output device to the cranium of a patient while the scanning operation is underway; focus the energy generated by the scanning device into the patient's cranium; and interconnect the power source, the scanning device, the data processing device, and the output device.

2. The apparatus of claim 1 wherein the number of scanning devices is two or more.

3. The apparatus of claim 1 wherein the scanning device is further comprising a transcranial doppler ultrasonography transducer array.

4. The apparatus of claim 3 wherein the number of scanning devices is two or more.

5. The apparatus of claim 1 wherein the scanning device is further comprising either a near infrared imager or an infrared imager transducer array.

6. The apparatus of claim 5 wherein the number of scanning devices is two or more.

7. A method of diagnosing conditions consistent with the existence of blockage of a patient's cranial blood vessels comprising the acts of:
a. mounting on a patient's cranium a head-mounted scanning tool capable of transmitting and receiving reflections from ultrasound waves, near infrared waves, infrared waves or similar technologies;
b. transmitting and receiving localizing signals over the patient's cranium to identify the optimal cranial mounting position;
c. broadcasting a single set of contemporaneous, synchronous pulses from the transmitter array into the patient's brain to perform an area scan of the patient's brain;
d. collecting data from reflected pulses via the receiver array and mapping the condition of blood flow within the brain;
e. analyzing collected data by comparing the data collected from a first portion of the patient's brain to that collected from a second portion of the patient's brain; and
f. interpreting the meaning of the collected data, developing a diagnosis based upon results of the analysis and outputting a diagnostic status signal to the output device.

8. The method according to claim 7 wherein a blockage of a patient's cranial blood vessels is a large vessel occlusion.

9. A method of diagnosing conditions consistent with the existence of blockage of a patient's cranial blood vessels comprising the acts of:
a. mounting the diagnostic tool of any of claims 1 through 6 on a patient's cranium;
b. transmitting and receiving localizing signals over the patient's cranium to identify the optimal cranial mounting position;
c. broadcasting a single set of contemporaneous, synchronous pulses from the transmitter array into the patient's brain to perform an area scan of the patient's brain;
d. collecting data from reflected pulses via the receiver array and mapping the condition of blood flow within the brain;
e. analyzing collected data by comparing the data collected from a first portion of the patient's brain to that collected from a second portion of the patient's brain; and
f. interpreting the meaning of the collected data, developing a diagnosis based upon results of the analysis and outputting a diagnostic status signal to the output device.

10. The method according to claim 9 wherein a blockage of a patient's cranial blood vessel is a large vessel occlusion.

11. A method of diagnosing and treating conditions consistent with the existence of blockage of a patient's cranial blood vessels comprising the acts of:
a. mounting on a patient's cranium a head-mounted scanning tool capable of transmitting and receiving reflections from ultrasound waves or similar technologies;
b. transmitting and receiving localizing signals over the patient's cranium to identify the optimal cranial mounting position;
c. broadcasting a single set of contemporaneous, synchronous pulses from the transmitter array into the patient's brain to perform an area scan of the patient's brain;
d. collecting data from reflected pulses via the receiver array and mapping the condition of blood flow within the brain;
e. analyzing collected data by comparing the data collected from a first portion of the patient's brain to that collected from a second portion of the patient's brain;
f. interpreting the meaning of the collected data, developing a diagnosis based upon results of the analysis and outputting a diagnostic status signal to the output device; and
g. treating an affected area by maintaining applied ultrasound energy to the location of the patient's brain wherein resides a suspected cranial blood vessel blockage.

12. The method according to claim 11 wherein a blockage of a patient's cranial blood vessel is a large vessel occlusion.

13. A method of diagnosing and treating conditions consistent with the existence of blockage of a patient's cranial blood vessels comprising the acts of:
a. mounting the diagnostic tool of claim 3 or claim 4 on a patient's cranium;
b. transmitting and receiving localizing signals over the patient's cranium to identify the optimal cranial mounting position;
c. broadcasting a single set of contemporaneous, synchronous pulses from the transmitter array into the patient's brain to perform an area scan of the patient's brain;
d. collecting data from reflected pulses via the receiver array and mapping the condition of blood flow within the brain;
e. analyzing collected data by comparing the data collected from a first portion of the patient's brain to that collected from a second portion of the patient's brain;
f. interpreting the meaning of the collected data, developing a diagnosis based upon results of the analysis and outputting a diagnostic status signal to the output device; and
g. treating an affected area by maintaining applied ultrasound energy to the location of the patient's brain wherein resides a suspected cranial blood vessel blockage.

14. The method according to claim 13 wherein a blockage of a patient's cranial blood vessel is a large vessel occlusion.
